(19) United States
(12) Patent Application Publication
(10) Pub. No.: US 2024/0384335 A1
(43) Pub. Date: Nov. 21, 2024

(54) LINKAGE ASSAY

(71) Applicant: Bio-Rad Laboratories, Inc., Hercules, CA (US)

(72) Inventors: Thomas Houser, Fulshear, TX (US); Andrew M. Prantner, Pleasanton, CA (US)

(21) Appl. No.: 18/666,949

(22) Filed: May 17, 2024

Related U.S. Application Data
(60) Provisional application No. 63/503,365, filed on May 19, 2023.

(51) Int. Cl. C12Q 1/6851; C12Q 1/6806; C12Q 1/686

(52) U.S. Cl. CPC C12Q 1/6851; C12Q 1/6806; C12Q 1/686

(57) ABSTRACT

The invention relates to methods for identifying, determining and quantifying linkages in fragments of nucleic acids.

### TECHNICAL FIELD

The invention generally refers to methods for quantifying linked target molecules and calculating higher order linkages in genetic variations.

### BACKGROUND

Genetic variation and protein dysfunction underly many aspects of disease. Measurement of genetic variation is important to several fields of research. For example, counting genetic changes in tumors can provide insight into the fundamental issues in cancer biology. Variations lie at the core of current problems in managing patients with viral diseases, such as AIDS and hepatitis, and to counteract drug resistance.

Genetic linkage analysis can prove to be a powerful tool to detect, for example, the location of genes on a chromosome. However, more specifically, genetic linkage analysis can be used to determine the relationship of genetic elements and their implications for disease. Linkage analysis can also determine whether the disease is monogenic or polygenic.

Genetic interactions resulting from linkage can contribute to several complex traits. Higher order linkages have a profound impact on human health and diseases. Higher order genetic interactions among three or more genes also plays a critical role in controlling phenotype. However, genetic interaction studies have been primarily focused on pairwise interactions involving two loci. Technical limitations have made conventional analysis of higher-order linkages difficult.

### SUMMARY OF THE INVENTION

The invention provides methods for quantifying and analyzing linked target nucleic acids in a biological sample. One aspect of the invention provides methods for quantifying genetic linkages related to variant analysis. Genetic variations may include any type of nucleic acid variation known in the art. Exemplary genetic variations include single nucleotide polymorphisms, rearrangements, substitutions, inversions, deletions and the like. The invention is also useful for the analysis of linkages between genetic elements, including epigenetic elements, that give rise to disease or increased risk of disease.

Aspects of the invention involve partitioning the biological sample into partitions, such as droplets, microwells and the like, molecular labeling, amplification, and counting the number of droplets that contain the linked target molecule of interest. A biological sample of the invention is any sample that contains genomic DNA. In some embodiments, partitioning of biological sample comprises emulsification of the biological sample containing genomic DNA into aqueous droplets such that the droplets are surrounded by immiscible fluid. Partitioning the biological sample into droplets or other partitions results in the majority of droplets having a single target molecule of interest. The target molecule of interest may be a linked or unlinked allelic variant.

In some embodiments, the biological sample containing genomic DNA is mixed with allele-specific fluorescence probes. Droplets are then generated using a droplet generator and transferred into, for example, 96 well plates. All DNA fragments from the biological sample are distributed into the droplets. A digital polymerase chain reaction is then conducted in the droplets. During the digital polymerase chain reaction, a fluorescence signal is generated based upon the contents of the droplet. The droplet may be positive for one unlinked target molecule, linked target molecules, two unlinked target molecules, etc. Some droplets may also be negative for the target molecules. Droplets are then read digitally for the presence of linked target molecules. Linkage is detected based on the observation that droplets positive for linked target molecules will give rise to double positive indicators in droplets. Double positive droplets emit a fluorescence signal different from droplets positive for single unlinked target molecules.

In an exemplary embodiment, the invention provides methods to quantify higher order linkages in a biological sample. To quantify copies per droplet of a species of N linked nucleic acid fragments, where N is the number of different sub-fragments that comprise the linked species, clusters of droplets positive for the species of N-linked nucleic acid fragments are selected. The value of N is from about two to about 10000. For example, if copies per droplet of a three-target linked species (ABC) is to be quantified, then clusters of droplets positive for the ABC three target linked species are selected. Then, clusters of droplets positive for fewer than three target-linked species are selected. For example, droplets positive for AB, BC, AC, A, B, C are also selected. To determine the number of copies of ABC per droplet (the three-target linked species), the following formula is used:

(Copies per droplet of total target A within all clusters)−(sum of A, AB, and AC)=X

(Copies per droplet of total target B within all clusters)−(sum of B, AB, and BC)=Y

(Copies per droplet of total target C within all clusters)−(sum of C, AC, and BC)=Z;

The average of X+Y+Z represents the copies per droplet of the three-target linked species (ABC).

Alternatively, the average can be a geometric average or an algorithm that optimizes a combination of linked species based on the theoretical error of each measurement. In addition, combinations can be weighted based on expected prevalence in the sample, error rates of measurement, and the number of linked species.

In one preferred embodiment, the invention comprises preparing a plurality of droplets, each comprising nucleic acid fragments obtained from a biological sample and selecting a species of N linked nucleic acid fragments for detection, wherein N is the number of different sub-fragments that comprise the linked species. Then, the droplets are assayed to identify or determine a subset of linked species that contain fewer than N linked sub-fragments, as well as the droplets that contain the individual (that make up the N total sub-fragments) sub-fragments alone. Finally, the N linked nucleic acid fragments are quantified as the average of a total for each sub-fragment less a total of linked species less than N comprising the sub-fragment. In alternative embodiments, the fragments are quantified as a weighted average.

In one example, droplet digital PCR technology is a digital PCR method utilizing a water-oil emulsion droplet system. Droplets are formed in a water-oil emulsion to form the partitions that separate the template DNA molecules. The droplets serve essentially the same function as individual test tubes or wells in a plate in which the PCR reaction takes place, albeit in a much smaller format. The massive sample partitioning is a key aspect of the ddPCR technique.

The Droplet Digital PCR System partitions nucleic acid samples into thousands of nanoliter-sized droplets, and PCR amplification is carried out within each droplet. This technique has a smaller sample requirement than other commercially available digital PCR systems, reducing cost and preserving precious samples. An exemplary system is, for example, a QX200 Droplet Digital PCR System (Bio-Rad, Hercules CA). The ddPCR workflow consists of combining a DNA sample and primers/probes in a supermix to create individual samples, generally about 20 ul in volume. The samples are loaded into a droplet generator to create a monodisperse emulsion. Prior to droplet generation, nucleic acid samples (DNA or RNA) are prepared as they are for any real-time assay: using primers, fluorescent probes (TaqMan probes with FAM and HEX or VIC), and a supermix developed specifically for droplet generation. Samples are then placed into a droplet generator, which utilizes proprietary reagents and microfluidics to partition the samples into about 20,000 nanoliter-sized droplets. The droplets ideally are uniform in size and volume.

In an alternative embodiment, protein interactions are measured according to the invention using linked oligonucleotide tags. In an embodiment of the invention, linked oligo sequences are associated with a binding element, such as an antibody, that bind specifically to a protein analyte in a sample. Determination of the linked species as presented herein reveal both the presence and quantitation of analytes available for detection.

The invention also comprises methods for detecting analytes or targets in a biological sample using linked nucleic acids. In one aspect linkages are used to encode sample origin. For example, a linked species, AB, may be associated with a particular sample of origin and BC, for example, with another sample of origin. In this way, downstream analysis can be multiplexed. In addition, quantitative linkage analysis is useful to uniquely identify analytes or classes of analytes in a sample. For example, particular linkage combinations of two or more linked sequences can be used to deconvolute target molecules in a sample.

### DETAILED DESCRIPTION

The invention generally relates to methods for quantifying linked target molecules in a biological sample. Linked target molecules are alleles that are present on the same fragment in a genomic DNA sample. Linkage analysis is important in the understanding of both normal and disease phenotypes.

Embodiments of the invention use Droplet Digital PCR (ddPCR) to enable quantification of linked target molecules. The ddPCR reaction involves mixing a biological sample with allele-specific fluorescence probes as well as reagents that are emulsified to form nanoliter-sized partitions, such that most partition comprises a single target species.

In an exemplary embodiment, the allele or variant specific fluorescent probes include but are not limited to FAM and HEX. For example, in case of two alleles located at different loci, FAM and HEX fluorophores act as a fluorescence reporter. The droplets also contain a supermix developed specifically for droplet generation and primers (e.g., ddPCR multiplex supermix, Bio-Rad, Hercules CA). The densely packed droplets are, for example, transferred to a 96 well PCR plate and thermal cycled to end-point. After the droplets are thermocycled, the plate is transferred to a droplet reader. Each droplet from each well is detected based on their fluorescence signal. Based on the fluorescence amplitude, a simple threshold assigns each droplet as positive or negative.

### Target

Methods of the present invention are used to quantify one or more linked target molecules. In some embodiments, the sample is a biological sample. Biological samples can be obtained from a number of biological organisms including but not limited to bacteria, fungus, plant, or any other organism. In some embodiments, the biological sample is from a mammal, for example, a human, a cow, a horse, a cat, a dog etc. A biological sample can be any tissue or body fluid.

Nucleic acid templates, including DNA, can be synthetic or derived from micro-organisms, such as bacteria or fungus, or from a human-beings, for example patients suffering from certain diseases. Nucleic acid templates can also be isolated from cultured cells, such as primary cell culture or a cell line. The nucleic acid templates are extracted from the cell lines by methods known in the art.

In some embodiments, two, three, four, or more different target molecules are detected. In some embodiments, the target molecules are N linked, where N is the number of sub-fragments that comprise the linked species. In some embodiments, the value for N may be about 3, 4, 5, 6, 7, 8, 9 or 10.

In some embodiments, the sample is prepared to improve the detection of the target molecule or target molecules. In an exemplary embodiment, the sample is fractionated. Droplet partitioning enables accurate quantitation of nucleic acid in the biological sample

In some embodiments, the sample is incubated with two or more probes prior to portioning the sample. In some embodiments, the two or more probes can be fluorescence probes and quencher for those probes.

Distribution into Partitions and Amplification

In the embodiments described herein, it is desirable to distribute and compartmentalize biological sample into partitions so that the partitions generally comprise at least one target nucleic acid to be amplified. In some embodiments, additional reagents may be added to the biological sample prior to partitioning.

In some embodiments, the compartmentalized portions are droplet-based emulsion systems. Compartmentalizing involves introducing the target nucleic acid sample into a stream of droplets. The emulsion comprises two immiscible liquids, for example, aqueous droplets in continuous oil phase. Preferred droplets for use in the invention are aqueous droplets surrounded by an immiscible carrier liquid. The immiscible carrier may be an oil. An emulsion of aqueous droplets is amenable for use in methods for conducting reactions with biological samples and detecting products may include a first fluid such as a water-based fluid. The water-based fluid is typically referred to as “aqueous” fluid. The water-based fluid may be suspended or dispersed as droplets within another fluid, preferably a hydrophobic fluid. The aqueous phase is also known as the discontinuous phase while the hydrophobic fluid is known as the continuous phase. The continuous phase may be a type of oil. Examples of oil that may be employed include, but are not limited to mineral oils, silicone-based oils, fluorinated oils.

Surfactants may be used to act as a stabilizer for emulsions. Surfactants may particularly be useful for embodiments that include conducting reactions with the biological samples such as PCR. Embodiments of surfactants may include one or more fluorinated or silicone surfactant. In microfluidic embodiments, the addition of one or more surfactants can aid in optimizing droplet size, flow, and uniformity. In some embodiments, aqueous droplets may be coated with a surfactant or a mixture of surfactants. Those of skill in the art understand that the surfactant molecules reside at the interface between immiscible fluids.

In some embodiments, the number of target nucleic acid molecules in the droplets is controlled by limiting dilution of the target nucleic acid molecules in the aqueous solution. In some embodiments, the number of target nucleic acid molecules in the droplets is controlled by partitioning very small volumes of the aqueous fluid into the droplet where the likelihood of distributing multiple target nucleic acid molecules in the same droplet is very small. In some embodiments, the distribution of molecules within droplets can be described by Poisson distribution.

Several methods of forming emulsions may be employed. In some embodiments, methods involve forming aqueous droplets in which some droplets contain no nucleic acid molecule and thus, no target molecule, while some droplets contain one target nucleic acid molecules, and a few droplets may contain multiple target nucleic acid molecules.

Droplets may be formed by any method known in the art. For example, droplets may be generated using an eight-channel droplet generator cartridge. The cartridge comprises of a droplet well, a sample well, and an oil well. The sample is added to the sample well and droplet generation oil is added to the oil well. Vacuum is applied to the droplet wells in the cartridge due to which sample and oil are drawn through a flow focusing nozzle where nano-liter sized monodisperse droplets are formed.

### Amplification Reaction in Partitions

For amplification, the sample may be pre-mixed with a primer of primers before droplet formation. In some embodiments, the droplets created by segmenting the starting sample are merged with a second set of droplets including one or more primers for the target nucleic acid in order to produce final droplets. In an exemplary embodiment, the ddPCR workflow comprises template, ddPCR Supermix for Probes (Bio-Rad Laboratories), primers, and fluorescence probes. Droplets are then generated using a droplet generator and then transferred into a 96-well plate. Droplet PCR amplification to end-point is performed in a conventional thermal cycler. Droplet PCR amplification gives rise to an amplification product in each droplet.

### Detecting Linked Target Molecules

In one embodiment, the biological sample is incubated with fluorescence probes wherein the probes are allele or variant specific. The probe specifically binds to the target molecule at the alleles, if present on the target molecule. In an exemplary embodiment, variant specific probes FAM and HEX are used to detect variants of interest. Following amplification as described above, the plate containing droplets is loaded onto a reader which sips droplets from each well and streams them past a multi-color detector. Droplets are designated as positive or negative based on their fluorescence amplitude. Droplets generated are either positive for FAM or HEX fluorophore. Some droplets generated by emulsification will also be positive for both FAM and HEX fluorophore while certain droplets will be negative for either FAM or HEX fluorophore. The droplets that are positive for both FAM and HEX may either have genetic variants that are physically linked to each other and thus on the same chromosome or may have unlinked genetic variants in the droplet due to chance co-localization. At high enough DNA loads, some droplets will contain both targeted and non-targeted genes. An example of non-targeted gene is the wild type sequence of the targeted gene that is present on the other chromosome. When both non-targeted and targeted genes are present in the same droplet, these templates compete for PCR reagents resulting in reduction of fluorescence amplitude for these droplets. At high enough DNA loads, competition can result in as many as three additional clusters (droplet populations) near each major FAM+, HEX+, and FAM+/HEX+ cluster. The only way to avoid additional clusters is to load less DNA.

In addition to competition, the probes also cross-react with non-targeted alleles, which can add extra clusters. Addition of non-fluorescent competitor probes that bind the non-targeted allele will greatly reduce cross reactivity.

In some embodiments, a digital readout assay can be used to count the number of target molecules by partitioning the target molecules in a sample and identifying the partitions containing the target. Generally, the process of digital readout assay involves determining for each partition of a sample whether the partition is positive or negative for target molecule. The partitions are examined for presence of two or more probes in each partition. The partition is “positive” for presence of target molecule if each of the two of more probes which were incubated with the sample are detected in the partition. For example, when the biological sample is incubated with FAM and HEX fluorescent probes, the droplets positive for both FAM and HEX are the droplets which positive for the presence of the target since the droplets have alleles that are linked. A partition is “negative” for the presence of the target molecule if only one of the probes or none of the probes are included in the droplets.

### Calculation of Higher-Order Linkages

Detection of linkage is based on the observation that the presence of linked target molecules will increase the number of double-positive droplets relative to the number expected due to chance. In order to calculate higher order linkages, the first step is to identify droplet clusters to quantify a given species of N-linked nucleic acid fragments, where N is the number of different sub-fragments that comprise the linked species. This collection contains clusters positive for zero or more of A, B, and/or C. Clusters positive for any other target (e.g., D, E, etc.) are not included.

Therefore, the method for determining copies per droplet of ABC triple linked molecule is:

(Copies per droplet of total target A within all clusters)−(sum copies per droplet of A, AB, and AC)

(Copies per droplet of total target B within all clusters)−(sum copies per droplet of B, AB, and BC)

(Copies per droplet of total target C within all clusters)−(sum copies per droplet of C, AC, and BC)

The value obtained for each result is in theory the same, but due to clustering error, the results may differ. An average, weighted average, geometric mean, or an error-based clustering analysis is performed for the values computed. Total target means all instances of target regardless of whether it appears on a linked species.

What is claimed is:

1. A method for quantifying linked molecules, the method comprising the steps of:
preparing a plurality of droplets, each comprising nucleic acid fragments obtained from a biological sample;
selecting a species of N linked nucleic acid fragments for detection, wherein N is the number of different sub-fragments that comprise the linked species;
assaying the droplets to determine a subset of linked species that contain fewer than N linked sub-fragments, and a subset of each of the sub-fragments alone; and
quantifying the N linked nucleic acid fragments as the average of a total for each sub-fragment less a total of linked species less than N comprising the sub-fragment.

2. The method of claim 1, wherein the quantifying step comprises a digital polymerase chain reaction.

3. The method of claim 2, wherein the digital polymerase chain reaction is conducted in the droplet.

4. The method of claim 3, wherein the droplet is an aqueous droplet surrounded by an immiscible fluid.

5. The method of claim 1, wherein the quantifying step comprises a digital readout obtained from a thermocycler in which the digital polymerase chain reaction takes place.

6. The method of claim 1, wherein N is from about 3 to about 10.

7. The method of claim 1, wherein the sub-fragments are contiguous on a single fragment in the sample.

8. The method of claim 1, wherein the sub-fragments are on the same fragment but separated by an intervening sequence.
